(19) United States
(12) Patent Application Publication
(10) Pub. No.: US 2017/0325744 A1
(43) Pub. Date: Nov. 16, 2017

(54) SYSTEMS AND METHODS FOR INCREASING LOCALIZED PRESSURE TO IMPROVE PPG MOTION PERFORMANCE

(71) Applicant: Apple Inc., Cupertino, CA (US)

(72) Inventors: Nicholas Paul Joseph ALLEC, Menlo Park, CA (US); Paul D. MANNHEIMER, Los Altos, CA (US); Tobias J. HARRISON-NOONAN, San Francisco, CA (US); Ueyn L. BLOCK, Menlo Park, CA (US); Vivek VENUGOPAL, San Jose, CA (US)

(21) Appl. No.: 15/592,020

(22) Filed: May 10, 2017

Related U.S. Application Data
(60) Provisional application No. 62/397,791, filed on Sep. 21, 2016.
(60) Provisional application No. 62/334,363, filed on May 10, 2016.

(51) Int. Cl. A61B 5/00; A61B 5/024; G02B 27/30; G02B 3/08

(52) U.S. Cl. CPC A61B 5/6843; A61B 5/681; A61B 5/6898; A61B 5/02427; A61B 2562/146; A61B 2562/0238; A61B 2562/0219; A61B 2562/185; G02B 3/08; G02B 27/30; A61B 2562/046

(57) ABSTRACT

The relates to a back surface of the device including one or more protrusions configured to create the localized pressure. In some examples, the protrusion(s) can be located between the optical components and one or more edges of the back plate. In some examples, the protrusion(s) can include a surface that can be raised relative to the back plate of the device. In some examples, one or more protrusions can include one or more recessed regions. In some examples, the cover structure disposed over each of the openings may itself be a protrusion that can apply local regions of higher pressure. The protrusion(s) can be capable of applying localized pressure to multiple spatially separated regions of the skin. Additionally or alternatively, the protrusion(s) can be capable of applying different amounts of localized pressure. Examples of the disclosure can include the Fresnel lens(es) and/or optical isolation optically coupled to the protrusion.

### CROSS-REFERENCE TO RELATED APPLICATIONS

This application claims priority to U.S. Provisional Patent Application Ser. No. 62/397,791 filed on Sep. 21, 2016 and U.S. Provisional Patent Application Ser. No. 62/334,363 filed on May 10, 2016, which are hereby incorporated by reference in their entirety.

### FIELD

This relates to architectures for PPG systems, and more specifically, to PPG systems configured to increasing localized pressure for improving PPG motion performance and methods for operation thereof.

### BACKGROUND OF THE DISCLOSURE

An individual's physiological signals (e.g., pulse rate or arterial oxygen saturation) can be determined by photoplethysmogram (PPG) systems. In a basic form, PPG systems can employ one or more light sources that can illuminate an individual's tissue and one or more light detectors that can receive light that enters and probes a subsurface volume of tissue. The received light can include light with an amplitude that can be modulated in time as a result of interaction with pulsatile blood flow and parasitic, non-signal light that can indirectly sample pulsatile tissue volumes with an amplitude that can be modulated (i.e., “noise” or “artifacts”) and/or unmodulated (i.e., DC).

### SUMMARY OF THE DISCLOSURE

This relates to systems and methods for increasing localized pressure to one or more skin regions of an individual. Applying localized pressure to the individual's skin, can lead to increased pulsatile signal, reduced local venous blood volume, and decreased venous contributions to motion artifacts for improved measurement accuracy of the individual's physiological information. The back surface of the device can include one or more protrusions configured to create the localized pressure. In some examples, the protrusion(s) can be located between the optical components (e.g., light sensors and/or light emitters) and one or more edges of the back plate. In some examples, the protrusion(s) can include a surface that can be raised (e.g., forming a plateau surface) relative to the back plate of the device. In some examples, one or more protrusions can include one or more recessed regions. In some examples, the cover structure disposed over each of the openings may itself be a protrusion that can apply local regions of higher pressure directly to the skin regions located in the optical path(s) of the light emitter(s) and/or light sensor(s). The protrusion(s) can be capable of applying localized pressure to multiple (e.g., two) spatially separated regions of the individual's skin. Additionally or alternatively, the protrusion(s) can be capable of applying different amounts of localized pressure. Examples of the disclosure can include the Fresnel lens(es) and/or optical isolation optically coupled to the protrusion.

### BRIEF DESCRIPTION OF THE DRAWINGS

FIGS. 1A-1C illustrate systems in which examples of the disclosure can be implemented

FIG. 2A illustrates a top view, and FIG. 2B illustrates a cross-sectional view of an exemplary electronic device including light sensors and light emitters for measuring an individual's physiological signal according to examples of the disclosure.

FIGS. 3A-3B illustrate perspective and cross-sectional views of an exemplary back surface of a device including a protrusion located between the optical components and one or more edges of the back plate according to examples of the disclosure.

FIGS. 4A-4B illustrate perspective and cross-sectional views of an exemplary back surface of a device including a protrusion having a plateau according to examples of the disclosure.

FIGS. 5A-5B illustrate perspective and cross-sectional views of an exemplary back surface including a recess associated with each cavity and opening of the device according to examples of the disclosure.

FIG. 5C illustrates a perspective view of an exemplary back surface of a device including a protrusion having multiple recesses associated with each cavity according to examples of the disclosure.

FIGS. 6A-6B illustrate perspective and cross-sectional views of an exemplary back surface of a device including cover structures that form the protrusions according to examples of the disclosure.

FIG. 6C illustrates an exemplary method of applying localized pressure to one or more skin regions of the individual according to examples of the disclosure.

FIG. 6D illustrates a cross-sectional view of an exemplary back surface of a device including cover structures that include protrusions and cover structures that do not include protrusions according to examples of the disclosure.

FIG. 7A illustrates a cross-sectional view of an exemplary protrusion according to examples of the disclosure.

FIGS. 7B-7C illustrate cross-sectional views of exemplary protrusions including a Fresnel lens located between the protrusion and cover structure according to examples of the disclosure.

FIGS. 8A-8B illustrate a cross-sectional view of exemplary protrusions including an isolation according to examples of the disclosure.

FIGS. 9A-9B illustrate cross-sectional and top views of an exemplary cover structure optically coupled to a plurality of light emitters, an isolation, and a Fresnel lens having multiple optical centers according to examples of the disclosure.

FIG. 9C illustrates a top view of an exemplary cover structure optically coupled to a plurality of light emitters, an isolation, and a patterned Fresnel lens according to examples of the disclosure.

FIG. 9D illustrates an exemplary method of applying localized pressure to one or more skin regions of the individual using a device including one or more Fresnel lenses according to examples of the disclosure.

FIGS. 10A-10B illustrate perspective and cross-sectional views of an exemplary back surface of a device including a monolithic cover structure according to examples of the disclosure.

FIG. 10C illustrates a perspective view of an exemplary back surface of a device including a monolithic cover structure that includes protrusions and non-monothilic cover structures that do not include protrusions according to examples of the disclosure.

FIG. 11 illustrates an exemplary block diagram of a computing system comprising back of cover touch sensor configurations according to examples of the disclosure.

### DETAILED DESCRIPTION

In the following description of examples, reference is made to the accompanying drawings in which it is shown by way of illustration specific examples that can be practiced. It is to be understood that other examples can be used and structural changes can be made without departing from the scope of the various examples.

Various techniques and process flow steps will be described in detail with reference to examples as illustrated in the accompanying drawings. In the following description, numerous specific details are set forth in order to provide a thorough understanding of one or more aspects and/or features described or referenced herein. It will be apparent, however, to one skilled in the art, that one or more aspects and/or features described or referenced herein may be practiced without some or all of these specific details. In other instances, well-known process steps and/or structures have not been described in detail in order to not obscure some of the aspects and/or features described or referenced herein.

Further, although process steps or method steps can be described in a sequential order, such processes and methods can be configured to work in any suitable order. In other words, any sequence or order of steps that can be described in the disclosure does not, in and of itself, indicate a requirement that the steps be performed in that order. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modification thereto, does not imply that the illustrated process or any of its steps are necessary to one or more of the examples, and does not imply that the illustrated process is preferred.

This relates to systems and methods for increasing localized pressure to one or more skin regions of an individual. Applying localized pressure to the individual's skin, can lead to increased pulsatile signal, reduced local venous blood volume, and decreased venous contributions to motion artifacts for improved measurement accuracy of the individual's physiological information. The back surface of the device can include one or more protrusions configured to create the localized pressure. In some examples, the protrusion(s) can be located between the optical components (e.g., light sensors and/or light emitters) and one or more edges of the back plate. In some examples, the protrusion(s) can include a surface that can be raised (e.g., forming a plateau surface) relative to the back plate of the device. In some examples, one or more protrusions can include one or more recessed regions. In some examples, the cover structure disposed over each of the openings may itself be a protrusion that can apply local regions of higher pressure directly to the skin regions located in the optical path(s) of the light emitter(s) and/or light sensor(s). The protrusion(s) can be capable of applying localized pressure to multiple (e.g., two) spatially separated regions of the individual's skin. Additionally or alternatively, the protrusion(s) can be capable of applying different amounts of localized pressure. Examples of the disclosure can include the Fresnel lens(es) and/or optical isolation optically coupled to the protrusion.

A user's physiological signals (e.g., pulse rate and arterial blood oxygen saturation) can be determined by measurements using pulse oximetry systems. Such pulse oximetry systems can be designed to be sensitive to changes in the red blood cell number, concentration, volume, or blood oxygen state included in the sample or a user's vasculature. In a basic form, pulse oximetry systems can employ a light source that injects light into the user's tissue and a light detector to receive light that reflects and/or scatters and exits the tissue. In some examples, at least a portion of the photon path length interacts with tissue subsurface structures. Pulse oximetry systems can include, but are not limited to, PPG systems and SpO2 systems. PPG and SpO2 systems can determine signals based on the attenuation of light over time. Attenuation can due to absorption, and/or scattering resulting from physiological/mechanical changes. Physiological/mechanical changes can include, but are not limited to, red blood cell number, cell/blood volume, red blood cell orientation, red blood cell/blood velocity, shear force, location/spatial distribution, or a combination thereof.

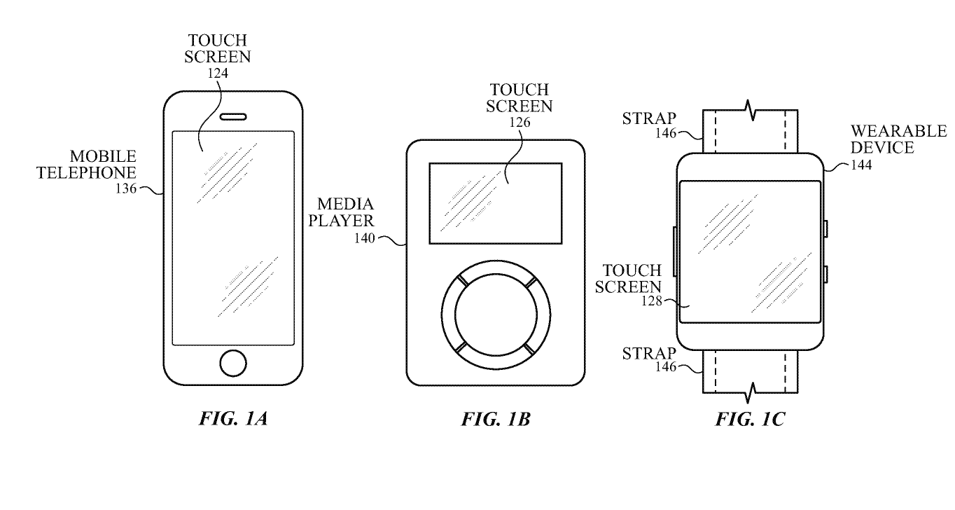

FIGS. 1A-1C illustrate systems in which examples of the disclosure can be implemented. FIG. 1A illustrates an exemplary mobile telephone 136 that can include a touch screen 124. FIG. 1B illustrates an exemplary media player 140 that can include a touch screen 126. FIG. 1C illustrates an exemplary wearable device 144 that can include a touch screen 128 and can be attached to an individual using a strap 146. The systems of FIGS. 1A-1C can include systems and methods for increasing localized pressure, as will be disclosed.

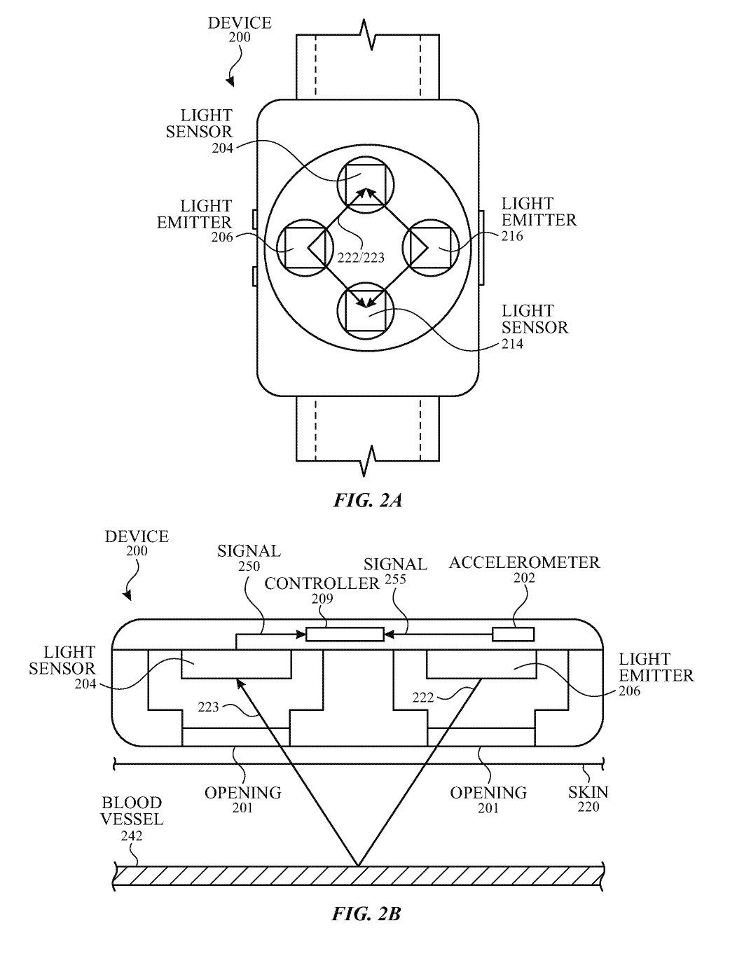

FIG. 2A illustrates a top view, and FIG. 2B illustrates a cross-sectional view of an exemplary electronic device including light sensors and light emitters for measuring an individual's physiological signal according to examples of the disclosure. The top view in FIG. 2A can be viewed as the underside of a wearable device (e.g., wearable device 144 of FIG. 1C). Device 200 can include light sensor 204, light sensor 214, light emitter 206, and light emitter 216. Light sensor 204 can be optically coupled to light emitter 206 and light emitter 216. Light sensor 214 can be optically coupled to light emitter 206 and light emitter 216. Device 200 can be situated such that light sensor 204, light sensor 214, light emitter 206, and light emitter 216 are proximate to the skin 220 of an individual. For example, device 200 can be held in an individual's hand or strapped to an individual's wrist, among other possibilities.

Light emitter 206 can be configured to emit light (e.g., light 222), included in one or more light rays, through opening 201. A portion of the one or more light rays can be absorbed by one or more blood vessels 242, and a portion of the one or more light paths can reflect back through opening 201 to be detected by a light sensor. For example, as illustrated in FIG. 2B, a portion of light 222 (emitted by light emitter 206) can be absorbed by blood vessel 242, and a portion of light (e.g., light 223) can reflect back for detection by light sensor 204. Light emitter 206 can also be configured to emit light, and a portion of light can reflect back for detection by light sensor 214. Similarly, light emitter 216 can be configured to emit light towards light sensor 204 and light sensor 214.

Light sensor 204 can be configured to generate signal 250. Signal 250 can include the measured total signal (e.g., sum of the measured modulated light and unmodulated light) detected by the light sensor (e.g., light sensor 204). In some examples, the device or system can include an accelerometer 202. Accelerometer 202 can be any type of sensor capable of measuring acceleration and can be configured to generate signal 255 indicative of the measured acceleration. Device 200 can include a processor or controller 209 configured to determine the individual's physiological signal from signal 250 and signal 255. The individual's physiological signal can be determined using any number of algorithms or simple mathematical functions including, but not limited to, subtracting, multiplying, and/or scaling.

In some instances, the signal (e.g., signal 250) can include noise due to motion artifacts, for example. As the individual moves, internal motion (e.g., the skin, vasculature, and other parts of the body expanding and contracting) can contribute to the motion artifacts. To improve motion performance, localized pressure can be created at the individual's skin by the one or more protrusions of the device. The one or more protrusions can be, for example, one or more rigid structures. In some examples, the applied pressure can be directly situated in the optical path (formed by the light emitter(s) and light sensor(s)). By applying localized pressure to the individual's skin, the pressure gradient across arterial walls can be reduced, which can lead to an increase in pulsatile (AC) signal. Additionally, the localized pressure can allow blood to mobilize out of the high-pressure region(s), which can reduce the local venous volume. Venous blood can be non-pulsatile blood that can absorb light, leading to reduced signal levels. In some instances, the DC signal measured by the light sensor(s) can increase, and the venous contributions to motion artifacts can be reduced. With increased pulsatile (DC) signal levels, the measured signal can include less noise, and signal-to-noise ratios can increase. Further, with increased pulsatile signal levels, the power consumption of the device can decrease without compromising measurement accuracy.

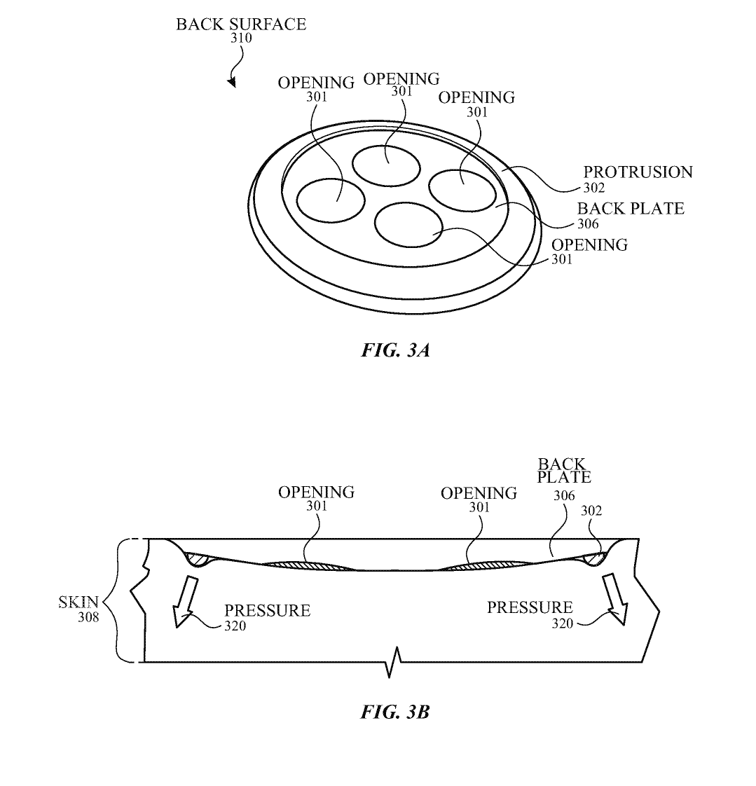

The device can include a back plate located on the back surface of the device. The back plate can include one or more structures (e.g., rigid structures) configured to create localized pressure (i.e., pressure in one or more regions of the individual's skin, where the area of the pressurized region can be smaller than the area of the back surface and/or back plate of the device). The one or more structures can include one or more protrusions having area(s) smaller than the area of the back plate. The one or more protrusions can be, for example, one or more rigid structures. In some examples, the one or more structures can include one or more protrusions located between the optical components (e.g., light sensors and/or light emitters) and one or more edges of the back plate. FIGS. 3A-3B illustrate perspective and cross-sectional views of an exemplary back surface of a device including a protrusion located between the optical components and one or more edges of the back plate according to examples of the disclosure. Back surface 310 of the device can include back plate 306 and protrusion 302. Protrusion 302 can be configured to create pressure to skin 308. Protrusion 302 can apply a greater amount of pressure 320 in a surrounding region (e.g., circular region) than the pressure created at other regions (e.g., region under openings 301) of skin 308.

Protrusion 302 can at least partially surround openings 301. In some examples, openings 301 can be configured to allow light emitted from a light emitter (e.g., light emitter 206 illustrated in FIG. 2A) to pass through to skin 308 and/or can allow light reflected from skin 308 to pass through to a light sensor (e.g., light sensor 204 illustrated in FIG. 2A). One or more optical components (e.g., light emitter(s), light sensor(s), or a combination thereof) can be located within the housing of the device and can be aligned with an opening 301 of a corresponding cavity. A transparent or translucent cover structure can be disposed over or within each of the openings 301 or cavities.

In some examples, protrusion 302 can be the same shape as back plate 306. For example, protrusion can be ring-shaped, which can include an open or closed ring (e.g., located around all edges of the device). In some examples, the protrusion can be arc-shaped. In some examples, protrusion 302 can surround all of openings 301 and corresponding cavities. Alternatively, back surface 310 can include a first protrusion that can surround a first set of cavities and a second protrusion that can surround a second set of cavities. In some examples, the protrusion may not surround or enclose any of the cavities, but instead can span a length or width of back surface 310 (not shown) (e.g., a rectangular protrusion parallel to one or more edges of the device).

Protrusion 302 can be disposed back plate 306. Protrusion 302 can protrude out from back surface 310 and/or back plate 306 such that pressure 320 can be created at skin 308 in one or more regions surrounding protrusion 302. Back plate 306 can be, for example, one or more rigid structures. In some examples, protrusion 302 can be configured to create localized pressure 320 to multiple (e.g., two) spatially separated regions of skin 308, as illustrated in FIG. 3B; wherein multiple optical components and/or the optical paths can be located between the spatially separated regions. In some examples, back plate 306 can have a convex curvature (as illustrated in FIG. 3B) or a concave curvature (not shown). In some examples, back plate 306 may not have any curves and may be substantially flat. In some examples, protrusion 302 can be embedded or disposed on back plate 306. In some examples, protrusion 302 can be a separate structure that can be adhered (e.g., using an adhesive) to back plate 306 and/or can protrude from back plate 306.

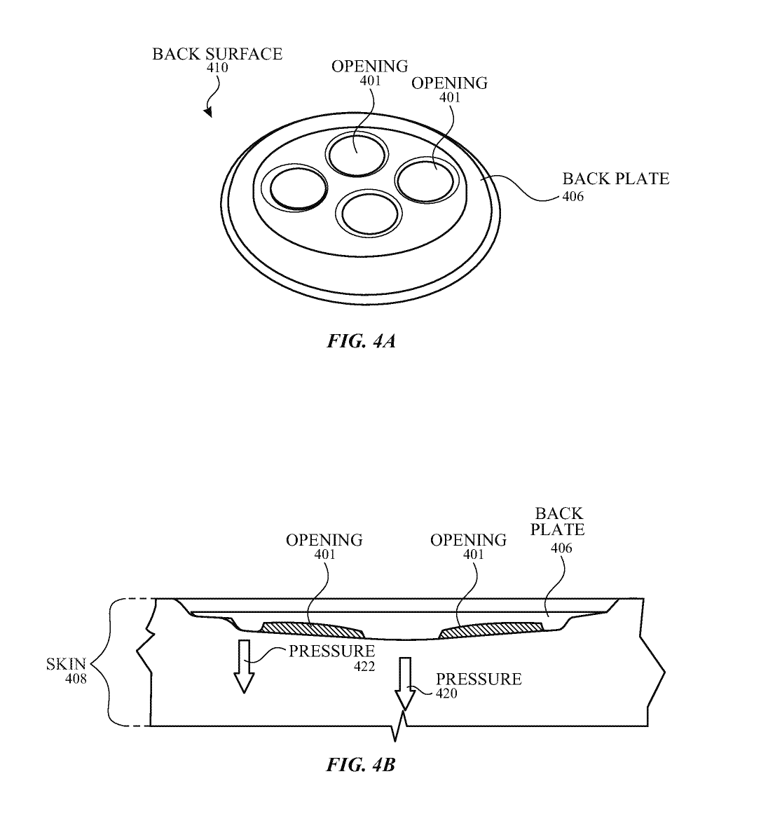

In some examples, pressure can be applied closer to the optical paths of the optical components and/or over a larger region(s) of the individual's skin. FIGS. 4A-4B illustrate perspective and cross-sectional views of an exemplary back surface of a device capable of creating multiple levels of localized pressure using a back plate according to examples of the disclosure. Back surface 410 can include back plate 406 and a plurality of openings 401. The plurality of openings 401 can be configured to allow light emitted from a light emitter (e.g., light emitter 206 illustrated in FIG. 2A) to pass through to skin 408 and/or can allow light reflected from skin 408 to pass through to a light sensor (e.g., light sensor 204 illustrated in FIG. 2A). One or more optical components (e.g., light emitter(s), light sensor(s), or a combination thereof) can be located within the housing of the device and can aligned with an opening 401 of a corresponding cavity. A transparent or translucent cover structure (e.g., window) can be disposed over or within each of the openings 401 or cavities.

Back plate 406 can include a plurality of sections that extend from back surface 410, where the plurality of sections can have different heights. In some examples, the surface of back plate 406 can be flat (i.e., without any curvature or curves) (not shown). In some examples, the surface of back plate 406 can have a convex curvature, as illustrated in FIG. 4B. The curvature can create the different heights such that multiple levels of localized pressure (e.g., pressure 420 and pressure 422) can be created. The cover structures (e.g., windows) disposed over or within openings 401 and can be flush with the surface of back plate 406 where back plate 406 joins (or forms) the cover structures. In some examples, the cover structures can protrude even further from the surface of back plate 406. In some examples, one or more cover structures can be separate and distinct from the back plate, and an adhesive, for example, can be used to adhere the cover structure(s) to the back plate and/or back surface. In some examples, one or more cover structure(s) can be integrated into the back plate.

Although FIG. 4A illustrates back plate 406 as having a circular shape, examples of the disclosure can include a back plate with any shape (e.g., ellipse, oval, rectangle, etc.). In some examples, back surface 410 can include two or more raised regions or protrusions that can be co-located with openings 401. For example, a back surface can include a first semi-circular protrusion that can extend over portions of the back surface that include a subset of one or more cavities and/or corresponding openings. The back surface can also include another subset of one or more cavities and/or corresponding openings. Examples of the disclosure can further include one or more cover structures that include an isolation, as discussed below.

The cavities (including light emitter(s) and/or light sensor(s) and corresponding openings) can be located at least partially on back plate 406. For example, back plate 406 can form a plateau that can extend from the edges of the device to the openings 401. In some examples, at least a portion of the corresponding cavities can be located on the surface of the plateau. Back plate 406 can extend over or across a substantial portion of the area of the back surface (e.g., the surface area of the plateau can be about 30%, 40%, 50%, 60% or more of the surface area of the entire back surface).

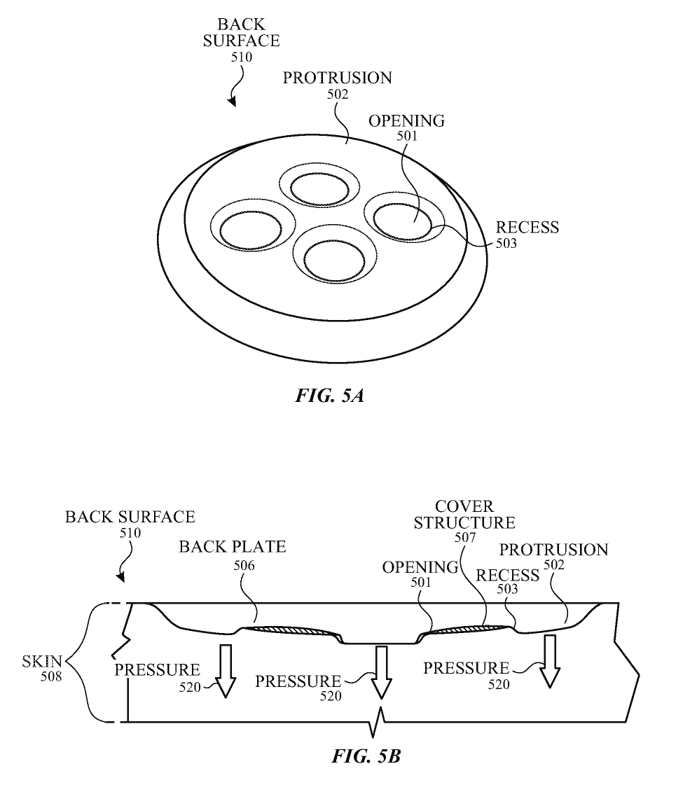

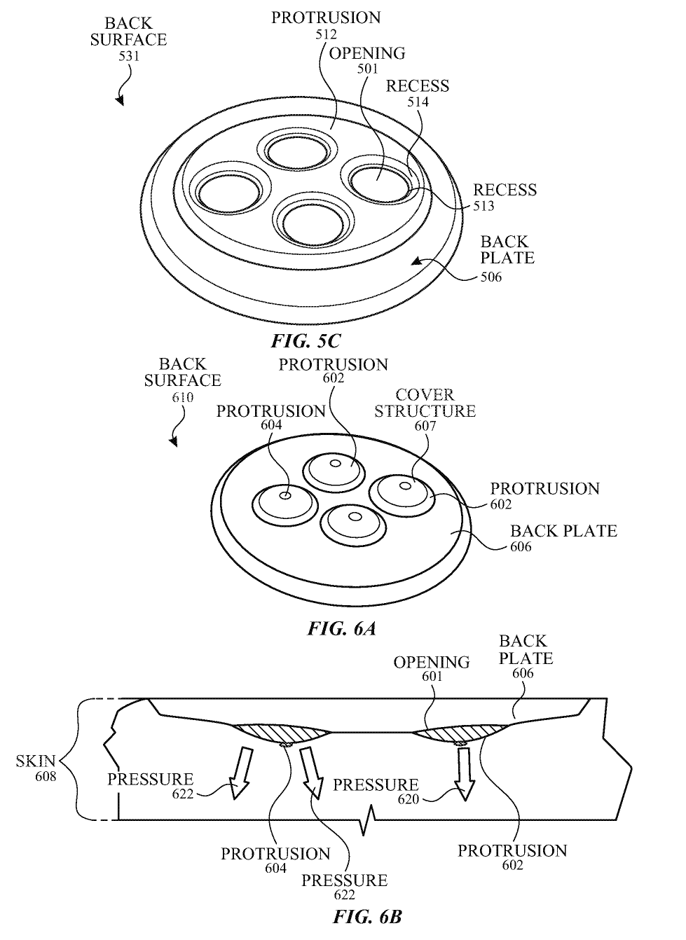

In some examples, one or more protrusions can include one or more recessed regions, the one or more recessed regions can include the opening and/or cover structures. FIGS. 5A-5B illustrate perspective and cross-sectional views of an exemplary back surface including a recess associated with each cavity and opening of the device according to examples of the disclosure. Back surface 510 can include protrusion 502, which can include recesses (i.e., recessed regions) 503. Recesses 503 can be each located over opening 501. That is, one or more cavities and corresponding light emitter(s) and/or light sensor(s) can be located within recess 503.

A cover structure 507 can be located within each cavity. In some examples, cover structure 507 can be set within each recess 503 such that the cover structure 507 may not be flush with (i.e., does not extend beyond) the surface of protrusion 502. In some examples, cover structure 507 can also be recessed from protrusion 502. Protrusion 502 can create greater levels of pressure 520 to skin regions compared to recesses 503, openings 501, and/or cover structures 507. In this manner, protrusion 502 can be configured to create localized pressure 320 to multiple (e.g., three) spatially separated regions of skin 308, as illustrated in FIG. 5B; wherein the spatially separated regions can include at least one region located between the optical components.

In some examples, the protrusion including recesses can have less surface area. In some examples, the device can be capable of applying multiple levels of localized pressure. FIG. 5C illustrates a perspective view of an exemplary back surface of a device including a protrusion having multiple recesses associated with each cavity according to examples of the disclosure. Back surface 531 can include protrusion 512. Protrusion 512 can include recess 513 and recess 514. Recess 513 can have a first depth (relative to the back surface of the back plate), and recess 514 can have a second depth (relative to the back surface of the back plate), the second depth being less than the first depth. Due to the differences in depths, protrusion can apply a greater amount of pressure to the individual's skin than either of the recesses. Recess 514 can apply a greater amount of pressure to the individual's skin than recess 513. In some examples, the surface area of protrusion 512 can be about 20% less, 30% less, 40% less, or 50% less than the surface area of protrusion 502 illustrated in FIGS. 5A-5B. In some examples, cover structure 507 can be recessed with respect to the back surface of the back plate, as shown in FIG. 5B. In some examples, one or more cover structures can be separate and distinct from the back plate, and an adhesive, for example, can be used to adhere the cover structure(s) to the back plate and/or back surface. In some examples, one or more cover structure(s) can be integrated into the back plate.

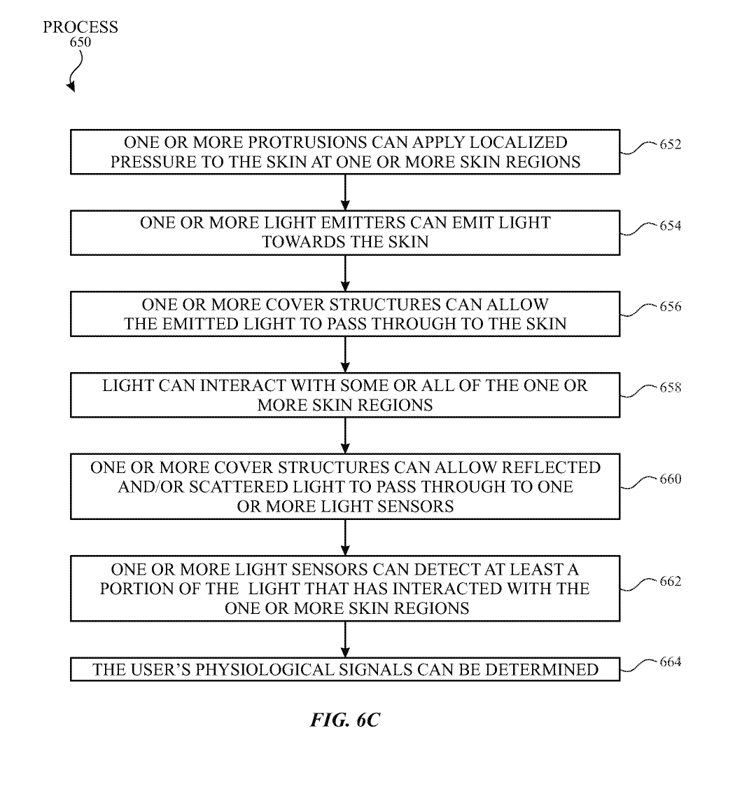

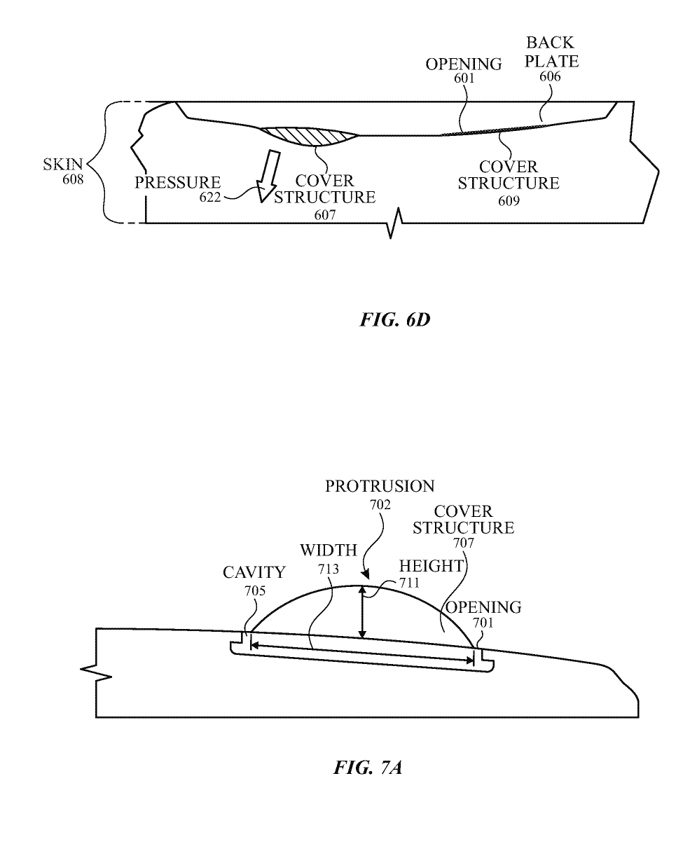

For example, the back surface can include one or more cavities having a corresponding opening and a protrusion located over each of the openings. In some examples, the cover structure disposed over each of the openings may itself be a protrusion that can apply local regions of higher pressure directly to the skin regions located in the optical path(s) of the light emitter(s) and/or light sensor(s). In other words, the skin region(s) that may be subject to increased levels of pressure may co-localize with the illumination field(s) of the one or more light emitter(s) and/or the field-of-view(s) of the one or more sensors. In some examples, the cover structures themselves can form protrusions. FIGS. 6A-6B illustrate perspective and cross-sectional views of an exemplary back surface of a device including cover structures that form the protrusions according to examples of the disclosure. Back surface 610 can include openings 601 and cover structures 607. Cover structures 607 can protrude from the surface of back plate 606. Back plate 606 can be, for example, one or more rigid structures. Cover structures 607 can include an optically transparent or translucent material such as acrylic, glass, and the like.

In some examples, multiple levels of localized pressure can be applied by the cover structures. Cover structures 607 can include one or more protrusions: protrusion 602 and, optionally, protrusion 604, where protrusion 602 can have a lower height (relative to the outward surface (i.e., surface facing skin 508 and/or external surface of the housing of the device) of the cover structure) than protrusion 604. In some examples, protrusion 604 can be located in the optical center of cover structure 607. The one or more protrusions (including the cover structure themselves) can be, for example, one or more rigid structures. In some examples, one or more cover structures can be separate and distinct from the back plate, and an adhesive, for example, can be used to adhere the cover structure(s) to the back plate and/or back surface. In some examples, one or more cover structure(s) can be integrated into the back plate.

Skin regions located under protrusion 602 can be subject to greater amounts of pressure 622 compared to skin regions located under non-protruding (e.g., back plate 606) portions of back surface 610. The radius of curvature of cover structures 602 can be consistent across the surface of the protrusion 602 (i.e., the curvature of a protrusion can approximate the curvature of a sphere). In some examples, the radius of curvature of cover structures 602 can vary (i.e., the curvature of a protrusion can be similar to the curvature of an oval). Skin regions located under protrusion 604 can be subject to greater amounts of pressure 620 compared to skin regions located under protrusion 602. In this manner, the localized pressure 620 and 622 can both be located in the optical path and/or field-of-view of the light emitter(s) and/or light sensor(s). In some examples, back plate can include one or more protrusions having a third height, where the third height can be less than the height of protrusion 602 and protrusion 604. That is, the back surface of the device can include three protrusions, each having different heights and configured to create different amounts of pressure.

FIG. 6C illustrates an exemplary method of applying localized pressure to one or more skin regions of the individual according to examples of the disclosure. One or more protrusions (e.g., protrusion 302 illustrated in FIGS. 3A-3B, back plate 406 illustrated in FIGS. 4A-4B, protrusion 502 illustrated in FIGS. 5A-5B, protrusion 512 illustrated in FIG. 5C, and protrusion 602 illustrated in FIGS. 6A-6B) can apply localized pressure (e.g., pressure 320 illustrated in FIG. 3B, pressure 420 illustrated in FIG. 4B, pressure 520 illustrated in FIG. 5B, and pressure 620 and pressure 622 illustrated in FIG. 6B) to the individual's skin (e.g., skin 220 illustrated in FIG. 3B, skin 308 illustrated in FIG. 3B, skin 408 illustrated in FIG. 4B, skin 508 illustrated in FIG. 5B, and skin 608 illustrated in FIG. 6B) at one or more skin regions (step 652 of process 650).

In some examples, the localized pressure can be created at multiple spatially separated regions of the individual's skin. In some examples, the localized pressure can be created at regions of the individual's skin located outside of the optical paths and/or field-of-view of the light emitter(s) and/or light sensor(s). In some examples, the localized pressure can be created at regions of the individual's skin in the optical paths and/or field-of-view of the light emitter(s) and/or light sensor(s). In some examples, the applied localized pressure can include different amounts of localized pressure. In some examples, the different amounts of localized pressure can be created by a protrusion disposed on the back plate, one or more recesses associated with a cavity, and/or one or more protrusions disposed on a cover structure.

One or more light emitters (e.g., light emitter 206 and light emitter 216 illustrated in FIG. 2A) can emit light towards the individual's skin (step 654 of process 650). One or more cover structures (e.g., cover structure 507 illustrated in FIG. 5B and cover structure 607 illustrated in FIG. 6A) can allow the emitted light to pass through to the individual's skin (step 656 of process 650). Light can interact with some or all of the one or more skin regions (step 658 of process 650) and can reflect and/or scatter back to the device. One more cover structures can allow reflected and/or scattered light to pass through to one or more light sensors (e.g., light sensor 204 and light sensor 214 illustrated in FIG. 2A) (step 660 of process 650). One or more light sensors can detect at least a portion of the light that has interacted with the one or more skin regions (step 662 of process 650). A processor or controller (e.g., controller 209) can determine the individual's physiological signals (step 664 of process 650).

The device can include any number of protrusions coupled to the one or more cover structures. In some example, some, but not all, of the cover structures can be associated with a protrusion. FIG. 6D illustrates a cross-sectional view of an exemplary back surface of a device including cover structures that include protrusions and cover structures that do not include protrusions according to examples of the disclosure. The device can include cover structure 607 and cover structure 609. Cover structure 607 can include a protrusion to create localized pressure in one or more regions of skin 608. Cover structure 609 may not include a protrusion and may not create localized pressure. Cover structure 609 can be flush (i.e., not protrude) from back plate 606. In some examples, cover structure 609 can be recessed with respect to back plate 606. In some examples, one or more cover structures can be separate and distinct from the back plate, and an adhesive, for example, can be used to adhere the cover structure(s) to the back plate and/or back surface. In some examples, one or more cover structure(s) can be integrated into the back plate.

Examples of the disclosure can include cover structure 607 optically coupled to a different type of optical component than cover structure 609 can be optically coupled to. For example, cover structure 607 can be optically coupled to one or more light emitters, whereas cover structure 609 can be optically coupled to one or more light sensors.

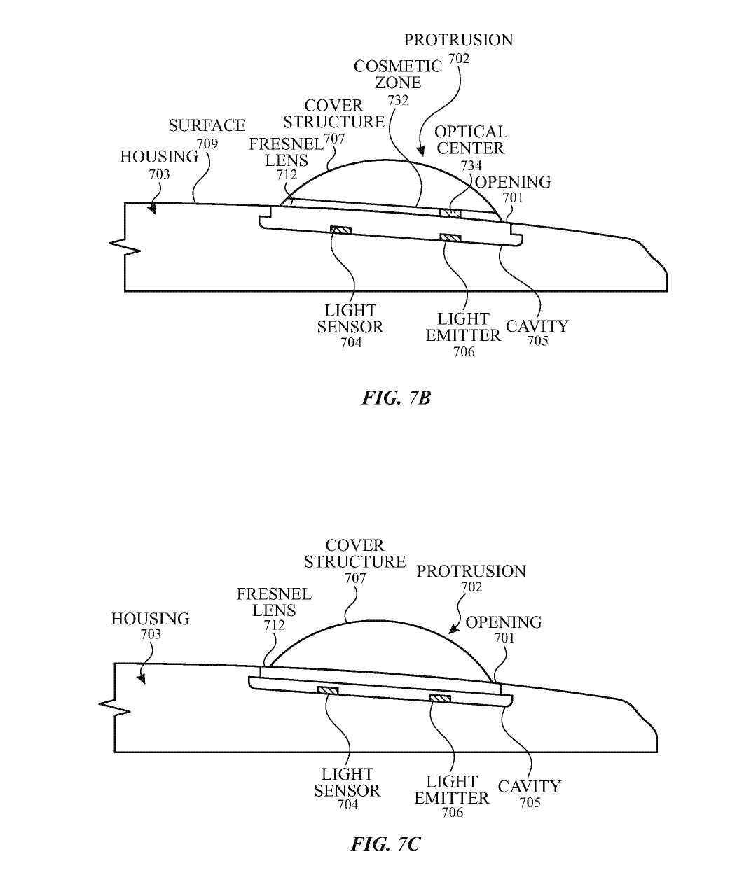

In some examples, the height and/or curvature of one or more protrusions on the back surface of a device can vary, as may be desired, to attain a desired pressure profile in the individual's skin. FIG. 7A illustrates a cross-sectional view of an exemplary protrusion according to examples of the disclosure. Protrusion 702 can be located over opening 701 of a cavity 705. In some examples, protrusion 702 can include the cover structure 707 (e.g., window) disposed over opening 701. Protrusion 702 can have a height 711 that can be between 0.3-2 mm. In some examples, height 711 can be 0.5 mm, 0.9 mm, 1.1 mm, or 1.3 mm. Protrusion 702 can have a radius of curvature that can be between 2.5-8.5 mm. In some examples, the radius of curvature can be 3.23 mm, 3.43 mm, 4.25 mm, 4.47 mm, 6.4 mm, or 7.47 mm. Protrusion 702 can have a base width 713 that can span the width of opening 701. In some examples, base width 713 can be less than the width of opening 701. In some examples, base width 713 can be between 3-10 mm. In some examples, base width 713 can be 3.5 mm, 4.5 mm, 5.4 mm, 6 mm, 7.3 mm, or 8.8 mm.

In some examples, the cover structure and/or protrusion can include a Fresnel lens or a similar optical component. In some instances, it may be desirable to obscure the optical components (e.g., light sensor(s) and/or light emitter(s)) and to reduce perceptibility of the optical components by an individual. In addition to obscuring internal components, it may be desirable for light emitted by the light emitter to retain its optical power, collection efficiency, beam shape, and collection area such that the light undergoes minimal change due to the cover structure. Examples of the disclosure can include the Fresnel lens(es) located in the protrusion.

In some examples, the Fresnel lens(es) can be located between the protrusion and cover structure, as illustrated in FIG. 7B. Fresnel lens 712 can be located between protrusion 702 and light emitter 706, which can be located in cavity 705. Fresnel lens 712 can be located above opening 701 of cavity 705, where cavity 705 can be recessed from the surface 709 of housing 703. Fresnel lens 712 can have multiple regions, such as an optical center 734 and a cosmetic zone 732. Optical center 734 can be placed in a same region or location as light emitter 706 and can be configured to collimate light emitted by light emitter 706 into a smaller beam size, for example. Cosmetic zone 732 can be located in regions outside of optical center 734. Cosmetic zone 732 can include one or more ridges to help obscure the underlying internal components.

Optionally, a light sensor 704 can be optically coupled to Fresnel lens 712. In some examples, light sensor 704 can be disposed in the same cavity 705 as light emitter 706. In some examples, light sensor 704 can be optically coupled to a different Fresnel lens (than light emitter 706). The different Fresnel lens may not have an optical center (e.g., because a light sensor may be a large area photodiode that may not require shaping of the light field), but can include a cosmetic zone having one or more ridges. The ridges can include, for example, saw tooth patterns, cylindrical ridges, asymmetric shapes, and wavy shape (i.e., ridges that move in an out).

Fresnel lens 712 may be used, additionally or alternatively, for light collimation. By collimating light, the optical efficiency can be improved. Without a lens or similar collimating optical element, emitted light may be directed at an angle away from the light sensor and can be lost. In some examples, light may be directed at an angle toward the light sensor, but the angle may be shallow (e.g., less than 15°). Fresnel lens 712 can redirect light to one or more directions to prevent light from being lost or entering into the skin at shallow angles. Such redirected light can be collected instead of being lost and/or may militate against parasitic non-signal light, which can improve optical signal efficiency. In some examples, a diffusing agent can be used in addition to (or instead of) a Fresnel lens. A diffusing agent can be surrounding, touching, and/or covering one or more components of the light emitter. In some examples, diffusing agent can be a resin or epoxy that encapsulates the dies (or any other components) and/or wire bonds. Diffusing agent can be used to adjust the angle(s) of light emitted by the light emitter. By narrowing the beam of emitted light, more light can be collected by the lens and/or window, and a larger amount of light can be detected by the light sensor. In some examples, Fresnel lens(es) can be located within the opening 701 of cavity 705 (e.g., with housing 703), as illustrated in FIG. 7C.

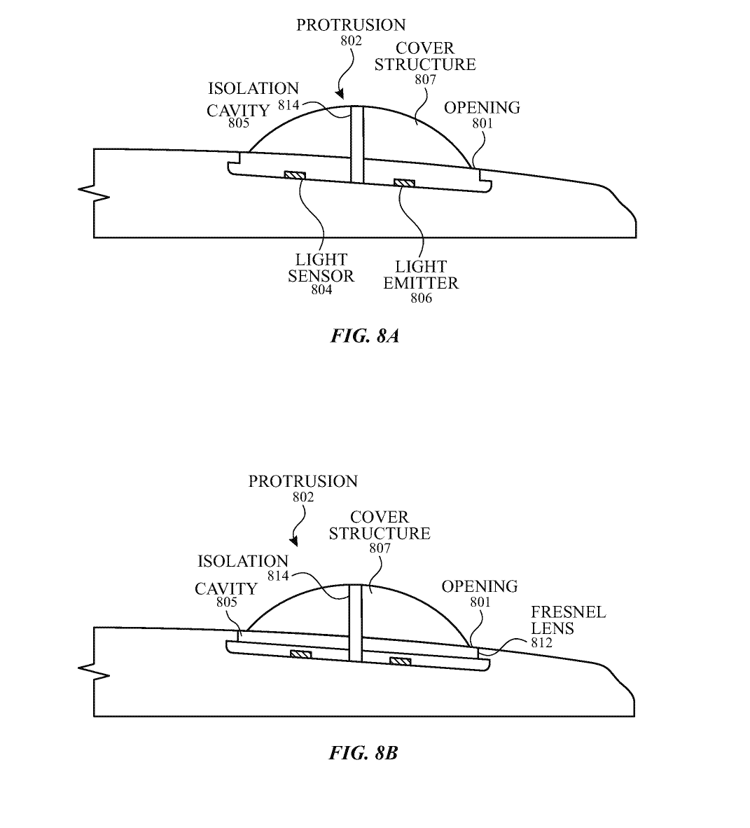

In some examples, one or more protrusions can include an isolation that can extend through the protrusion, where the isolation can be configured to separate light rays of the optical components on one side of the protrusion and/or cover structure from light rays on the other side. The isolation can extend from within the cavity, through the cavity, and/or through the protrusion. FIG. 8A illustrates a cross-sectional view of an exemplary protrusion including an isolation according to examples of the disclosure. Protrusion 802 can be disposed over opening 801 of cavity 805. Protrusion 802 can include an isolation 814, which can extend through the protrusion. In some examples, isolation 814 can extend inside (e.g., to the base of) cavity 805. While the figure illustrates isolation 814 as being substantially perpendicular to the base of cavity 805, examples of the disclosure can include isolation 814 having a non-perpendicular angle with respect to the base of cavity 805. In some examples, protrusion 802 can be included in cover structure 807, which can be coupled to light sensor 804 and light emitter 806. In some examples, protrusion 802 can include an isolation and can be optically coupled to a Fresnel lens, as illustrated in FIG. 8B. Fresnel lens 812 can include one or more designs and/or operation similar to Fresnel lens 712 illustrated in FIGS. 7B-7C and discussed above.

In some examples, the isolation can be located in the one or more cover structures, and the device can further comprise one or more separate structures including the one or more protrusions (having the same features, design, and/or operation as described above). In some examples, both the cover structures and the separate structures can include protrusions.

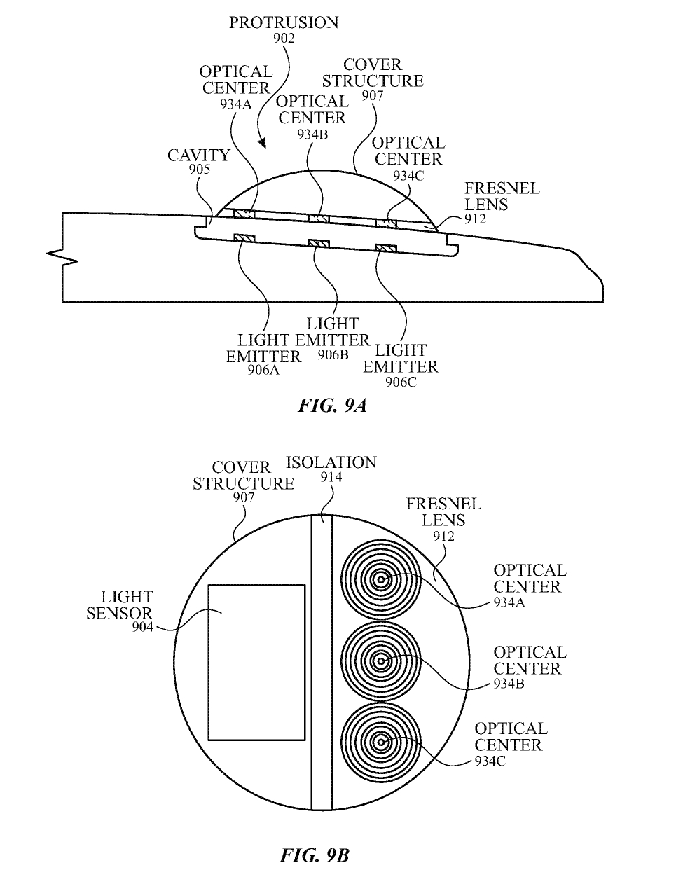

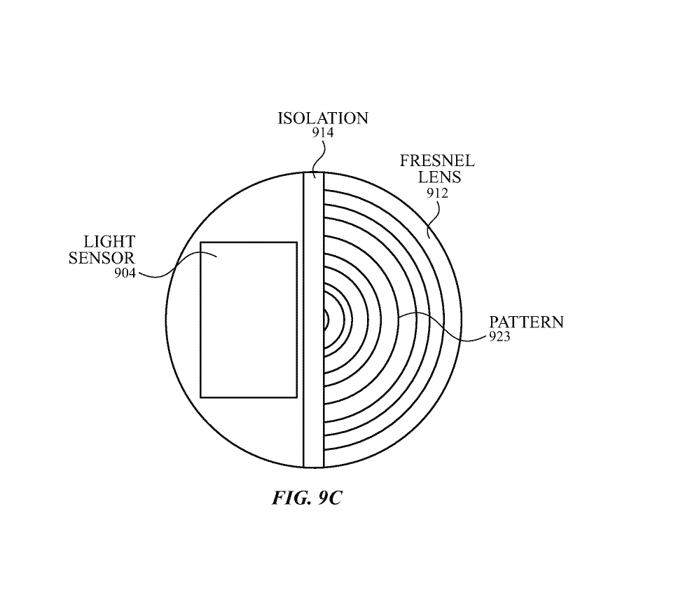

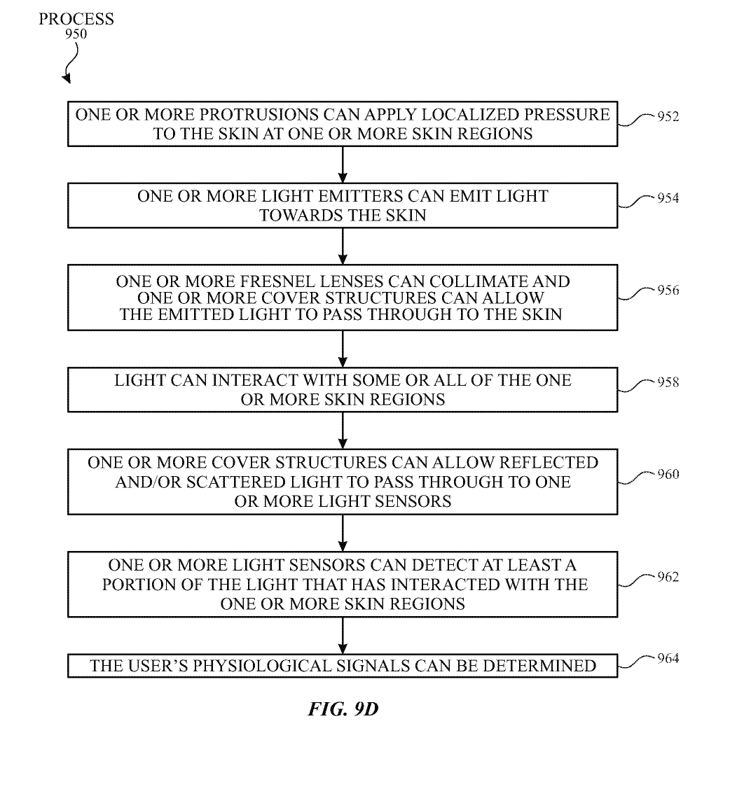

In some examples, the protrusion and/or cover structure can be optically coupled to a Fresnel lens having multiple optical centers. FIGS. 9A-9B illustrate cross-sectional and top views of an exemplary cover structure optically coupled to a plurality of light emitters, an isolation, and a Fresnel lens having multiple optical centers according to examples of the disclosure. Protrusion 902 can be included in cover structure 907. Cover structure 907 can be optically coupled to light emitter 906A, light emitter 906B, and light emitter 906C located in cavity 905. In some examples, light sensor 904 can be disposed in the same cavity 905 as the light emitters. In some examples, isolation 914 can provide an optical barrier between light sensor 904 and the plurality of light emitters (e.g., light emitter 906A, light emitter 906B, and light emitter 906C).

Fresnel lens 912 can be located between the plurality of light emitters and protrusion 902. Fresnel lens can include a plurality of optical centers, such as optical center 934A, optical center 934B, and optical center 934C. Optical center 934A can be optically coupled to light emitter 906A; optical center 934B can be optically coupled to light emitter 906B; and optical center 934C can be optically coupled to light emitter 906C. In some examples, the center of each optical center can be located over (e.g., aligned with) the center of its corresponding light emitter. In some examples, the ridge pattern of each optical center can include a plurality of concentric rings, spirals, semicircles, and/or arcs (e.g., pattern 923 illustrated in FIG. 9C). Examples of the disclosure can include one or more Fresnel lenses located in the cover structure, and/or underneath the housing (e.g., within the volume enclosed by the housing). In some examples, the plurality of light emitters may not be collinearly arranged (e.g., can be offset with respect to each other), and the optical centers of the Fresnel lens can be arranged to correspond to the positions of the plurality of light emitters.

FIG. 9D illustrates an exemplary method of applying localized pressure to one or more skin regions of the individual using a device including one or more Fresnel lenses according to examples of the disclosure. One or more protrusions (e.g., protrusion 302 illustrated in FIGS. 3A-3B, back plate 406 illustrated in FIGS. 4A-4B, protrusion 502 illustrated in FIGS. 5A-5B, protrusion 512 illustrated in FIG. 5C, protrusion 602 illustrated in FIGS. 6A-6B, protrusion 702 illustrated in FIGS. 7A-7C, protrusion 802 illustrated in FIGS. 8A-8B, and protrusion 902 illustrated in FIG. 9A) can apply localized pressure (e.g., pressure 320 illustrated in FIG. 3B, pressure 420 illustrated in FIG. 4B, pressure 520 illustrated in FIG. 5B, and pressure 620 and pressure 622 illustrated in FIG. 6B) to the individual's skin (e.g., skin 220 illustrated in FIG. 3B, skin 308 illustrated in FIG. 3B, skin 408 illustrated in FIG. 4B, skin 508 illustrated in FIG. 5B, and skin 608 illustrated in FIG. 6B) at one or more skin regions (step 952 of process 950).

In some examples, the localized pressure can be created at multiple spatially separated regions of the individual's skin. In some examples, the localized pressure can be created at regions of the individual's skin located outside of the optical paths and/or field-of-view of the light emitter(s) and/or light sensor(s). In some examples, the localized pressure can be created at regions of the individual's skin in the optical paths and/or field-of-view of the light emitter(s) and/or light sensor(s). In some examples, the applied localized pressure can include different amounts of localized pressure. In some examples, the different amounts of localized pressure can be applied by a protrusion disposed on the back plate, one or more recesses associated with a cavity, and/or one or more protrusions disposed on a cover structure.

One or more light emitters (e.g., light emitter 206 and light emitter 216 illustrated in FIG. 2A) can emit light towards the individual's skin (step 954 of process 950). One or more Fresnel lenses (e.g., Fresnel lens 712 illustrated in FIGS. 7B-7C, Fresnel lens 812 illustrated in FIG. 8B, and Fresnel lens 912 illustrated in FIGS. 9A-9C) and one or more cover structures (e.g., cover structure 507 illustrated in FIG. 5B, cover structure 607 illustrated in FIG. 6A, cover structure 707 illustrated in FIGS. 7B-7C, cover structure 807 illustrated in FIG. 8B, and cover structure 907 illustrated in FIG. 9A) can allow the emitted light to pass through to the individual's skin (step 956 of process 950). Light can interact with some or all of the one or more skin regions (step 958 of process 950) and can reflect and/or scatter back to the device. One more cover structures can allow reflected and/or scattered light to pass through to one or more light sensors (e.g., light sensor 204 and light sensor 214 illustrated in FIG. 2A) (step 960 of process 950). One or more light sensors can detect at least a portion of the light that has interacted with the one or more skin regions (step 962 of process 950). A processor or controller (e.g., controller 209) can determine the individual's physiological signals (step 964 of process 950).

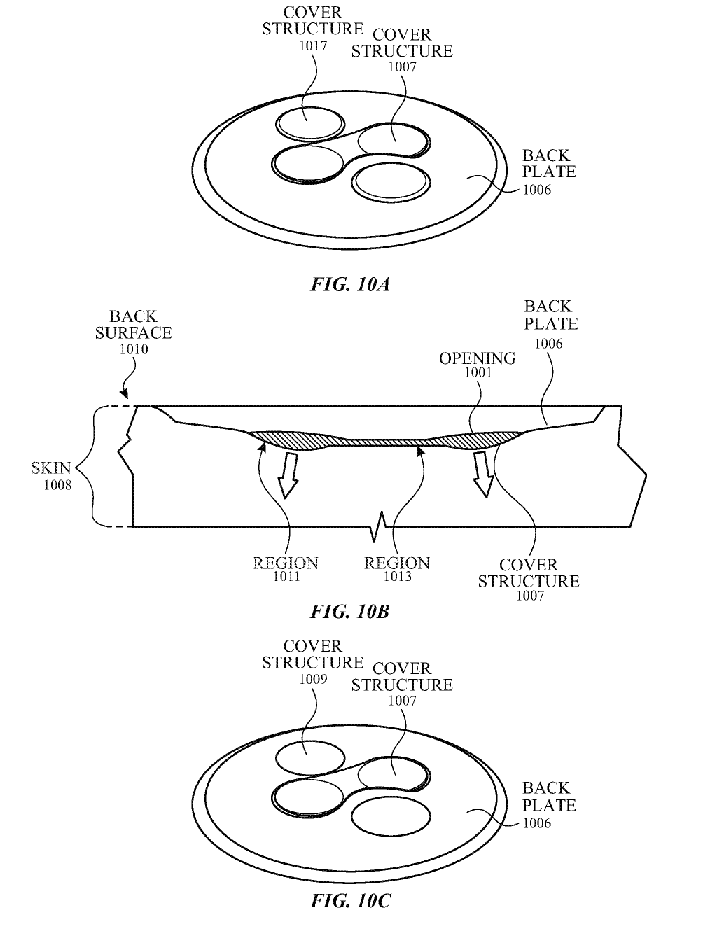

In some examples, one or more cover structures can be monolithic cover structures. FIGS. 10A-10B illustrate perspective and cross-sectional views of an exemplary back surface of a device including a monolithic cover structure according to examples of the disclosure. Back surface 1010 of the device can include back plate 1006 and a plurality of cover structures such as cover structure 1007 and cover structure 1017. Cover structure 1007 and cover structure 1017 can be transparent or translucent (e.g., a window) and can be disposed over or within one or more of the openings 1001 or cavities.

Each cover structure 1017 can be optically coupled to a single cavity (e.g., cavity 705 illustrated in FIGS. 7A and 7C and cavity 805 illustrated in FIGS. 8A-8B). Cover structure 1017 can have the same design, features, and/or operation as one or more of cover structure 507 (illustrated in FIG. 5B), cover structure 607 (illustrated in FIG. 6A), and cover structure 707 (illustrated in FIG. 7C). Cover structure 1007 can be a monolithic cover structure having one or more protrusions. For example, cover structure 1007 can have one elongated protrusion, protruding in regions 1011 located in the optical path(s) of the associated optical components and protruding in the region 1013. Region 1013 can be located between regions 1011 and/or disposed over a portion of the back plate located between a plurality of cavities. In some examples, the height (relative to an outward surface of the back plate) in regions 1011 and region 1013 can be the same (not shown). In some examples, cover structure 1007 can have multiple protrusions, protruding in both regions 1011 and region 1013, but with different heights. For example, regions 1011 can have the same height, greater than the height of region 1013. In some examples, each region 1011 can have a different height.

In some examples, cover structure 1007 can be optically coupled to multiple (e.g., two) cavities. In some examples, cover structure 1007 can be optically coupled to a different type of optical component(s) than the type of optical component(s) that cover structure 1017 can be optically coupled to. For example, cover structures 1017 can be optically coupled to light sensors, whereas cover structure 1007 can be optically coupled to light emitters. In some examples, region 1011 (i.e., region located between the optical path(s) of the associated optical components) may be transparent. In some examples, region 1011 can be opaque and/or include an isolation. In this manner, region 1011 can be configured to create localized pressure, while also optically isolating the optical components located in different cavities.

The device can include any number of protrusions coupled to the one or more cover structures. In some example, some, but not all, of the cover structures can be associated with a protrusion. FIG. 10C illustrates a perspective view of an exemplary back surface of a device including a monolithic cover structure that includes protrusions and non-monothilic cover structures that do not include protrusions according to examples of the disclosure. The device can include cover structure 1007 and covers structure 1009. Cover structure 1007 can include a protrusion to create localized pressure in one or more regions of skin. Cover structure 1009 may not include a protrusion and may not create localized pressure. Cover structure 1009 can be flush (i.e., not protrude) from back plate 1006. In some examples, cover structure 1009 can be recessed with respect to back plate 1006. In some examples, the cover structure (e.g., cover structure 1007) that includes one or more protrusions can be a monolithic cover structure. Additionally or alternatively, the cover structure (e.g., cover structure 1009) that does not include a protrusion may be a non-monolithic cover structure.

Examples of the disclosure can include cover structure 1007 optically coupled to a different type of optical component than cover structure 1009 can be optically coupled to. For example, cover structure 1007 can be optically coupled to one or more light emitters, whereas cover structure 1009 can be optically coupled to one or more light sensors.

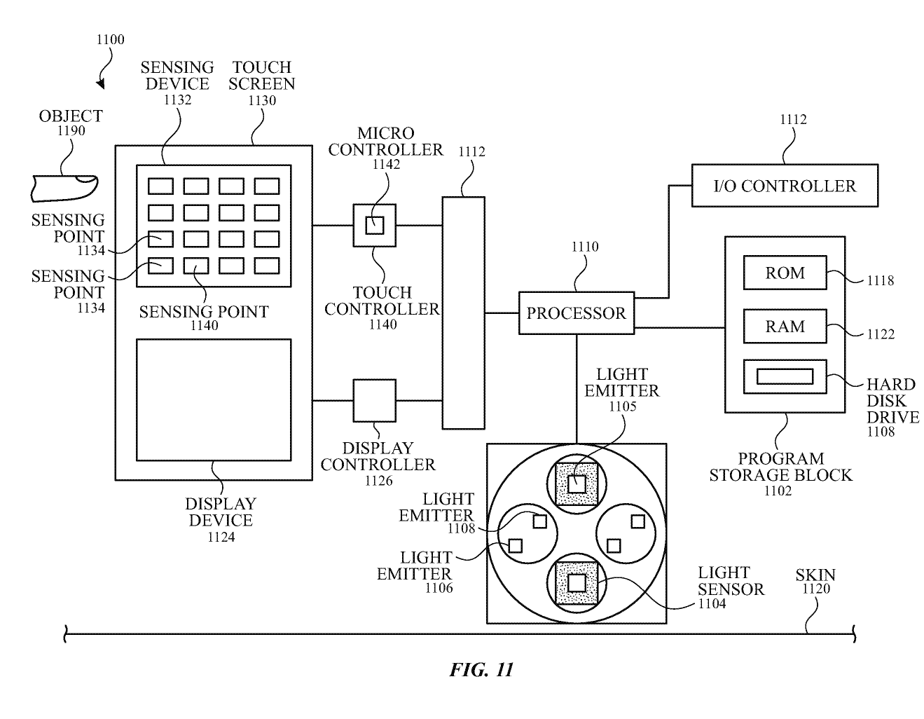

FIG. 11 illustrates an exemplary block diagram of a computing system comprising one or more protrusions for creating localized pressure according to examples of the disclosure. Computing system 1100 can correspond to any of the computing devices illustrated in FIGS. 1A-1C. Computing system 1100 can include a processor 1110 configured to execute instructions and to carry out operations associated with computing system 1100. For example, using instructions retrieved from memory, processor 1110 can control the reception and manipulation of input and output data between components of computing system 1100. Processor 1110 can be a single-chip processor or can be implemented with multiple components.

In some examples, processor 1110 together with an operating system can operate to execute computer code and produce and use data. The computer code and data can reside within a program storage block 1102 that can be operatively coupled to processor 1110. Program storage block 1102 can generally provide a place to hold data that is being used by computing system 1100. Program storage block 1102 can be any non-transitory computer-readable storage medium, and can store, for example, history and/or pattern data relating to PPG signals and/or physiological information of the individual measured by one or more light sensors (e.g., light sensor 1104), optically coupled to one or more light emitters (e.g., light emitter 1106, light emitter 1105, and light emitter 1108). In some examples, the system can include one or more protrusions located in the optical path(s) and/or field-of view of the one or more light sensors and/or the one or more light emitters.

By way of example, program storage block 1102 can include Read-Only Memory (ROM) 1118, Random-Access Memory (RAM) 1122, hard disk drive 1108 and/or the like. The computer code and data could also reside on a removable storage medium and loaded or installed onto the computing system 1100 when needed. Removable storage mediums include, for example, CD-RM, DVD-ROM, Universal Serial Bus (USB), Secure Digital (SD), Compact Flash (CF), Memory Stick, Multi-Media Card (MMC) and a network component.

Computing system 1100 can also include an input/output (I/O) controller 1112 that can be operatively coupled to processor 1110 or it may be a separate component as shown. I/O controller 1112 can be configured to control interactions with one or more I/O devices. I/O controller 1112 can operate by exchanging data between processor 1110 and the I/O devices that desire to communicate with processor 1110. The I/O devices and I/O controller 1112 can communicate through a data link. The data link can be a one-way link or a two way link. In some cases, I/O devices can be connected to I/O controller 1112 through wireless connections. By way of example, a data link can correspond to PS/2, USB, Firewire, IR, RF, Bluetooth or the like.

Computing system 1100 can include a display device 1124 that can be operatively coupled to processor 1110. Display device 1124 can be a separate component (peripheral device) or can be integrated with processor 1110 and program storage block 1102 to form a desktop computer (all in one machine), a laptop, handheld or tablet computing device of the like. Display device 1124 can be configured to display a graphical user interface (GUI) including perhaps a pointer or cursor as well as other information to the individual. By way of example, display device 1124 can be any type of display including a liquid crystal display (LCD), an electroluminescent display (ELD), a field emission display (FED), a light emitting diode display (LED), an organic light emitting diode display (OLED) or the like.

Display device 1124 can be coupled to display controller 1126 that can be coupled to processor 1110. Processor 1110 can send raw data to display controller 1126, and display controller 1126 can send signals to display device 1124. Data can include voltage levels for a plurality of pixels in display device 1124 to project an image. In some examples, processor 1110 can be configured to process the raw data.

Computing system 1100 can also include a touch screen 1130 that can be operatively coupled to processor 1110. Touch screen 1130 can be a combination of sensing device 1132 and display device 1124, where the sensing device 1132 can be a transparent panel that is positioned in front of display device 1124 or integrated with display device 1124. In some cases, touch screen 1130 can recognize touches and the position and magnitude of touches on its surface. Touch screen 1130 can report the touches to processor 1110, and processor 1110 can interpret the touches in accordance with its programming. For example, processor 1110 can perform tap and event gesture parsing and can initiate a wake of the device or powering on one or more components in accordance with a particular touch.

Touch screen 1130 can be coupled to a touch controller 1140 that can acquire data from touch screen 1130 and can supply the acquired data to processor 1110. In some cases, touch controller 1140 can be configured to send raw data to processor 1110, and processor 1110 processes the raw data. For example, processor 1110 can receive data from touch controller 1140 and can determine how to interpret the data. The data can include the coordinates of a touch as well as pressure exerted. In some examples, touch controller 1140 can be configured to process raw data itself. That is, touch controller 1140 can read signals from sensing points 1134 located on sensing device 1132 and turn them into data that the processor 1110 can understand.

Touch controller 1140 can include one or more microcontrollers such as microcontroller 1142, each of which can monitor one or more sensing points 1134. Microcontroller 1142 can, for example, correspond to an application specific integrated circuit (ASIC), which works with firmware to monitor the signals from sensing device 1132, process the monitored signals, and report this information to processor 1110.

One or both display controller 1126 and touch controller 1140 can perform filtering and/or conversion processes. Filtering processes can be implemented to reduce a busy data stream to prevent processor 1110 from being overloaded with redundant or non-essential data. The conversion processes can be implemented to adjust the raw data before sending or reporting them to processor 1110.

In some examples, sensing device 1132 can be based on capacitance. When two electrically conductive members come close to one another without actually touching, their electric fields can interact to form a capacitance. The first electrically conductive member can be one or more of the sensing points 1134, and the second electrically conductive member can be an object 1190 such as a finger. As object 1190 approaches the surface of touch screen 1130, a capacitance can form between object 1190 and one or more sensing points 1134 in close proximity to object 1190. By detecting changes in capacitance at each of the sensing points 1134 and noting the position of sensing points 1134, touch controller 1140 can recognize multiple objects, and determine the location, pressure, direction, speed and acceleration of object 1190 as it moves across the touch screen 1130. For example, touch controller 1190 can determine whether the sensed touch is a finger, tap, or an object covering the surface.

Sensing device 1132 can be based on self-capacitance or mutual capacitance. In self-capacitance, each of the sensing points 1134 can be provided by an individually charged electrode. As object 1190 approaches the surface of the touch screen 1130, the object can capacitively couple to those electrodes in close proximity to object 1190, thereby stealing charge away from the electrodes. The amount of charge in each of the electrodes can be measured by the touch controller 1140 to determine the position of one or more objects when they touch or hover over the touch screen 1130. In mutual capacitance, sensing device 1132 can include a two-layer grid of spatially separated lines or wires, although other configurations are possible. The upper layer can include lines in rows, while the lower layer can include lines in columns (e.g., orthogonal). Sensing points 1134 can be provided at the intersections of the rows and columns. During operation, the rows can be charged, and the charge can capacitively couple from the rows to the columns. As object 1190 approaches the surface of the touch screen 1130, object 1190 can capacitively couple to the rows in close proximity to object 1190, thereby reducing the charge coupling between the rows and columns. The amount of charge in each of the columns can be measured by touch controller 1140 to determine the position of multiple objects when they touch the touch screen 1130.

A device is disclosed. The device can comprise: one or more optical components; one or more cover structures, each cover structure optically coupled to at least one of the one or more optical components and located on a back surface of the device; a rigid back plate extending from the back surface of the device; and one or more protrusions extending from the rigid back plate, wherein an area of the one or more protrusions is less than an area of the rigid back plate. Additionally or alternatively, in some examples, the one or more protrusions are located between the one or more optical components and one or more edges of the device. Additionally or alternatively, in some examples, the one or more protrusions form a closed ring located around all edges of the device. Additionally or alternatively, in some examples, the device further comprises: one or more openings in a housing of the device, wherein the one or more optical components and the one or more cover structures are located at least partially in the one or more openings, and the one or more protrusions at least partially surround the one or more openings. Additionally or alternatively, in some examples, the one or more protrusions are configured to create a localized pressure to one or more regions of a skin of an individual, and the one or more regions are located in an optical path of the one or more optical components. Additionally or alternatively, in some examples, the one or more protrusions are separate and distinct structures adhered to the rigid back plate. Additionally or alternatively, in some examples, the device further comprises: one or more recesses surrounded by the one or more protrusions; and one or more cavities located in a housing of the device, wherein the one or more optical components are located in the one or more cavities, and each recess is associated with at least one of the one or more cavities. Additionally or alternatively, in some examples, each cavity is associated with at least two recesses, the at least two recesses having different depths from a surface of the rigid back plate. Additionally or alternatively, in some examples, each cavity is associated with one of the one or more cover structures, and the one or more cover structures are recessed with respect to a surface of the rigid back plate. Additionally or alternatively, in some examples, the device further comprises: one or more second cover structures optically coupled to one or more light sensors, the one or more light sensors included in the one or more optical components, wherein the one or more cover structures include the one or more protrusions and are optically coupled to one or more light emitters, the light emitters included in the one or more optical components. Additionally or alternatively, in some examples, the rigid back plate has a curved surface. Additionally or alternatively, in some examples, the rigid back plate is separate and distinct from the one or more cover structures.

A device is disclosed. The device can comprise: one or more optical components; a rigid back plate located on a back surface of the device; and one or more cover structures, each cover structure optically coupled to at least one of the one or more optical components and located on the back surface, wherein each cover structure is at least partially transparent and protrudes from the rigid back plate. Additionally or alternatively, in some examples, at least one of the one or more cover structures includes a first protrusion having a first protrusion height from a surface of the rigid back plate and a second protrusion having a second protrusion height from the surface of the rigid back plate, the second protrusion height greater than the first protrusion height. Additionally or alternatively, in some examples, the rigid back plate has a third protrusion height from the back surface of the device, wherein the third protrusion height is less than a height of the first protrusion from the back surface of the device. Additionally or alternatively, in some examples, the device further comprises: one or more Fresnel lenses optically coupled to the one or more cover structures. Additionally or alternatively, in some examples, at least one of the one or more Fresnel lenses includes multiple optical centers. Additionally or alternatively, in some examples, the one or more cover structures include an isolation configured to optically isolate light rays from optical components located on different sides of a given cover structure. Additionally or alternatively, in some examples, at least one cover structure is a monolithic cover structure optically coupled to a plurality of cavities and disposed over a portion of the rigid back plate located between the plurality of cavities. Additionally or alternatively, in some examples, the monolithic cover structure includes a plurality of regions, each region disposed over one of the plurality of cavities, wherein a height of each region is greater than a height of the cover structure disposed over the portion of the rigid back plate. Additionally or alternatively, in some examples, the one or more cover structures are integrated into the rigid back plate.

A method for determining one or more physiological signals of an individual is disclosed. The method can comprise: emitting light from one or more light emitters; allowing the emitted light to pass through one or more first cover structures; receiving at least a portion of the emitted light using one or more light sensors; creating a first localized pressure at one or more first regions of a skin of the individual; allowing the at least the portion of the emitted light to pass through one or more second cover structures; generating one or more signals indicative of the received light; and determining the one or more physiological signals of the individual from the one or more signals. Additionally or alternatively, in some examples, the localized pressure is created at multiple spatially separated first regions of the skin of the individual. Additionally or alternatively, in some examples, the method further comprises: creating a second localized pressure at one or more second regions of the skin of the individual, the second localized pressure greater than the first localized pressure. Additionally or alternatively, in some examples, the first localized pressure is created by the one or more first cover structures.

A method for determining one or more physiological signals of an individual is disclosed. The method can comprise: emitting light from one or more light emitters located on a first side of a cover structure; optically isolating the emitted light from a second side of the cover structure; allowing the emitted light to pass through the first side of the cover structure; allowing at least a portion of the emitted light to pass through the second side of the cover structure; optically isolating the at least the portion of the emitted light from the second side of the cover structure; receiving the at least the portion of the emitted light using one or more light sensors located on the second side of the cover structure; generating one or more signals indicative of the received light; and determining the one or more physiological signals of the individual from the one or more signals. Additionally or alternatively, in some examples, the method further comprises: collimating the emitted light using one or more Fresnel lenses.

Although examples have been fully described with reference to the accompanying drawings, it is to be noted that various changes and modifications will become apparent to those skilled in the art. Such changes and modifications are to be understood as being included within the scope of the various examples as defined by the appended claims.

1. A device comprising:
one or more optical components;
one or more cover structures, each cover structure optically coupled to at least one of the one or more optical components and located on a back surface of the device;
a rigid back plate extending from the back surface of the device; and
one or more protrusions extending from the rigid back plate, wherein an area of the one or more protrusions is less than an area of the rigid back plate.

2. The device of claim 1, wherein the one or more protrusions are located between the one or more optical components and one or more edges of the device.

3. The device of claim 2, wherein the one or more protrusions form a closed ring located around all edges of the device.

4. The device of claim 1, further comprising:
one or more openings in a housing of the device, wherein the one or more optical components and the one or more cover structures are located at least partially in the one or more openings, and the one or more protrusions at least partially surround the one or more openings.

5. The device of claim 1, wherein the one or more protrusions are configured to create a localized pressure to one or more regions of a skin of an individual, and the one or more regions are located in an optical path of the one or more optical components.

6. The device of claim 1, further comprising:
one or more recesses surrounded by the one or more protrusions; and
one or more cavities located in a housing of the device, wherein the one or more optical components are located in the one or more cavities, and each recess is associated with at least one of the one or more cavities.

7. The device of claim 6, wherein each cavity is associated with at least two recesses, the at least two recesses having different depths from a surface of the rigid back plate.

8. The device of claim 6, wherein each cavity is associated with one of the one or more cover structures, and the one or more cover structures are recessed with respect to a surface of the rigid back plate.

9. The device of claim 1, further comprising:
one or more second cover structures optically coupled to one or more light sensors, the one or more light sensors included in the one or more optical components, wherein the one or more cover structures include the one or more protrusions and are optically coupled to one or more light emitters, the light emitters included in the one or more optical components.

10. A device comprising:
one or more optical components;
a rigid back plate located on a back surface of the device; and
one or more cover structures, each cover structure optically coupled to at least one of the one or more optical components and located on the back surface, wherein each cover structure is at least partially transparent and protrudes from the rigid back plate.

11. The device of claim 10, wherein at least one of the one or more cover structures includes a first protrusion having a first protrusion height from a surface of the rigid back plate and a second protrusion having a second protrusion height from the surface of the rigid back plate, the second protrusion height greater than the first protrusion height.

12. The device of claim 11, wherein the rigid back plate has a third protrusion height from the back surface of the device, wherein the third protrusion height is less than a height of the first protrusion from the back surface of the device.

13. The device of claim 10, further comprising:
one or more Fresnel lenses optically coupled to the one or more cover structures.

14. The device of claim 10, wherein at least one of the one or more Fresnel lenses includes multiple optical centers.

15. The device of claim 10, wherein at least one cover structure is a monolithic cover structure optically coupled to a plurality of cavities and disposed over a portion of the rigid back plate located between the plurality of cavities.

16. The device of claim 15, wherein the monolithic cover structure includes a plurality of regions, each region disposed over one of the plurality of cavities, wherein a height of each region is greater than a height of the cover structure disposed over the portion of the rigid back plate.

17. A method for determining one or more physiological signals of an individual, the method comprising:
emitting light from one or more light emitters;
allowing the emitted light to pass through one or more first cover structures;
receiving at least a portion of the emitted light using one or more light sensors;
creating a first localized pressure at one or more first regions of a skin of the individual;
allowing the at least the portion of the emitted light to pass through one or more second cover structures;
generating one or more signals indicative of the received light; and
determining the one or more physiological signals of the individual from the one or more signals.

18. The method of claim 17, further comprising:
creating a second localized pressure at one or more second regions of the skin of the individual, the second localized pressure greater than the first localized pressure.

19. A method for determining one or more physiological signals of an individual, the method comprising:
emitting light from one or more light emitters located on a first side of a cover structure;
optically isolating the emitted light from a second side of the cover structure;
allowing the emitted light to pass through the first side of the cover structure;
allowing at least a portion of the emitted light to pass through the second side of the cover structure;
optically isolating the at least the portion of the emitted light from the second side of the cover structure;
receiving the at least the portion of the emitted light using one or more light sensors located on the second side of the cover structure;
generating one or more signals indicative of the received light; and
determining the one or more physiological signals of the individual from the one or more signals.

20. The method of claim 19, further comprising:
collimating the emitted light using one or more Fresnel lenses.
